(19) United States
(12) Patent Application Publication
(10) Pub. No.: US 2025/0169968 A1
(43) Pub. Date: May 29, 2025

(54) CONTROLLABLE ARTIFICIAL HAND SYSTEM

(71) Applicant: OZYEGIN UNIVERSITESI, Istanbul (TR)

(72) Inventors: Mahsa SARRAFIKHOSROWSHAH, Istanbul (TR); Nedime KARAKULLUKCU, Kayseri (TR); Fatih ALTINDIS, Kayseri (TR); Bulent YILMAZ, Kayseri (TR); Ramazan UNAL, Istanbul (TR)

(73) Assignee: OZYEGIN UNIVERSITESI, Istanbul (TR)

(21) Appl. No.: 18/839,743

(22) PCT Filed: Mar. 1, 2022
(86) PCT No.: PCT/TR2022/050183

§ 371 Date: Aug. 20, 2024

(30) Foreign Application Priority Data
Feb. 28, 2022 (TR) 2022/002849

(51) Int. Cl. A61F 2/72; A61F 2/50; A61F 2/58; A61F 2/70; A61F 2/76

(52) U.S. Cl. CPC A61F 2/72; A61F 2/585; A61F 2002/5056; A61F 2002/5093; A61F 2002/701; A61F 2002/704; A61F 2002/7635

(57) ABSTRACT

An artificial hand system can be controlled with an intelligent wrist configuration in order to reduce the physical and mental load/discomfort on the muscles of an individual using a hand prosthesis while using the said hand.

### CROSS REFERENCE TO THE RELATED APPLICATIONS

This application is the national phase entry of International Application No. PCT/TR2022/050183, filed on Mar. 1, 2022, which is based upon and claims priority to Turkish Patent Application No. 2022/002849, filed on Feb. 28, 2022, the entire contents of which are incorporated herein by reference.

### TECHNICAL FIELD

The invention relates to an artificial hand system that can be controlled with an intelligent wrist configuration in order to reduce the physical and mental load/discomfort on the muscles of an individual using a hand prosthesis while using the said hand.

### BACKGROUND

The human hand is a very complex system that contains many degrees of freedom, sensors, tendons, actuators and control mechanisms in its structure. The loss of this limb, which is used in most of the daily activities of the individual, leads to a decrease in interaction with the environment and quality of life. In the studies, it has been determined that even the loss of a thumb of the hand causes 50% of hand function to be lost. Today, hand prostheses are used to help eliminate the deficiency caused by hand loss. The hand prostheses used are divided into two as body-powered and active prostheses.

Considering the body-powered hand prosthesis, even performing simple tasks such as picking up small objects, using a computer mouse, which do not require a lot of body movement, requires great effort. In the prostheses used in the current art, the functionality of the biological wrist cannot be utilized while carrying an object. For this reason, the prosthesis user tries to move the upper part of the body too much. The simplicity, ease of use and structure that reduces physical effort have an important role in the acceptance of the prosthesis by individuals. On the other hand, a lightweight and functional wrist is always more beneficial as it provides low inertia.

In brain-machine interface (BMI) technology, the signals received from the brain are processed with mathematical and statistical approaches, and the commands that the individual wants to give are determined from the processed signals. In this way, external devices such as computer cursors, wheelchairs, robotic arm prostheses can be controlled with BMI.

Motor intention is defined as imagining an actual movement before performing this movement. Previous studies have proven that simply imagining a movement activates the regions (the sensorimotor cortex) of the brain responsible for producing the movement in a similar way to those during actual movement. Each of the waves obtained from the brain has a different frequency and amplitude value. The alpha wave is between 8-13 Hz and increases in situations where the mind is empty such as resting and meditation. Beta waves are the waves which are between 13-30 Hz and complex and low amplitude compared to alpha waves. Alpha and beta band activities of electroencephalography (EEG) waves obtained from the sensorimotor cortex at the moment of motor intention change. During motor intention, the amplitude of alpha waves decreases, and this is called event-related desynchronization (ERD). In contrast, the amplitude of beta waves increases during motor intention, and this is called event-related synchronization (ERS). It is possible to use these changes in alpha and beta waves to control prosthetic devices.

Intuitive control can be developed by obtaining the user's intention from the signals recorded by non-invasive methods (such as EEG, electromyography (EMG)). One of the main constraints of current BMIs is the lack of a reliable interface with high accuracy, in other words low false rate that can intuitively control the movement of the prosthesis with user intention. Therefore, BMIs using only EEG signals are insufficient to control bionic hand prostheses.

Another alternative is control systems developed by using EMG signals. Classical myoelectric control is based on on/off or proportional techniques. Although the said techniques are widely used in commercial and clinical applications, they do not allow the user to control more than one degree of freedom at the same time. In addition, these systems require a long training phase. In these systems, signal distortions may occur due to sweating or improper placement of the socket. Therefore, an intuitive and unnatural prosthesis control occurs for the individual. This situation causes a great mental load and alienation on the prosthesis user. EMG-based control systems receive signals from the muscles of the amputee limbs to control the prosthesis. However, the muscles weaken due to long-term inactivity and cannot provide sufficient signals to control the prosthesis. On the contrary, in cases where the muscles are used continuously, this causes muscle fatigue and causes shifts in the frequency content of the EMG signals towards low frequency components. As a result, EMG signals alone cannot provide the necessary data for prosthesis control.

Wrist movement is an important requirement for the functional use of the prosthetic hand in daily life activities. However, in the studies, wrist movement of the upper extremity prosthesis was performed using the healthy hand (passive) or rotation (active) by means of EMG signals. In the studies, it was determined that the fact that the prosthetic wrist is suitable only for rotation movement causes complaints in the musculoskeletal systems of the individuals.

As a result, all the problems mentioned above made it necessary to make an innovation in the related technical field.

### SUMMARY

The present invention relates to an artificial hand system that can be controlled with an intelligent wrist configuration in order to eliminate the above-mentioned disadvantages and to bring new advantages to the related technical field.

One object of the invention is to provide an artificial hand system that enables individuals to reduce the physical and mental load/discomfort they may experience while using a mechanical prosthesis.

Another object of the invention is to provide an artificial hand system that makes individuals feel the bionic hand prosthesis they use as their own limb.

In order to achieve all the objects mentioned above and which will emerge from the detailed description below, the present invention relates to an artificial hand system to reduce the physical and mental discomfort in the muscles of an individual using a hand prosthesis while using the said hand. Accordingly, it comprises a wrist configuration whose stiffness can be controlled according to the weight of a load; at least one sensing unit for sensing the weight of said load, a processor unit for receiving data from said sensing unit, at least one drive element actuated by the processor unit; the processor unit is configured to
receive at least one data related to the weight of the load from the sensing unit,
process at least one data about the weight taken from the sensing unit by methods such as signal processing and machine learning,
detect the continuous and/or gradual relationship of the processed data with the weight of the object,
generate an output signal containing the weight information of the load as a result of the detected weight relationship;
actuate the drive element to set the stiffness of the wrist configuration based on the output signal.

Thus, by processing the biological signals received from the body and the signals received from the devices placed on the individual, it is ensured that the amount of load to be lifted is predicted and the prosthetic hand system is prepared in accordance with this amount of load. By preconditioning the prosthetic hand system in accordance with the load to be lifted in advance, it will be ensured that the factors that cause physical and mental fatigue experienced by the individual due to the use of prosthesis while performing daily activities will be alleviated.

The feature of one possible embodiment of the invention is that the sensing unit comprises at least one first sensor for measuring biological activity signals in the brain.

The feature of another possible embodiment of the invention is that the sensing unit contains at least one second sensor for measuring the biological activity signals that occur due to muscle movement.

The feature of another possible embodiment of the invention is that the sensing unit comprises at least one third sensor for measuring visual signals.

The feature of another possible embodiment of the invention is that the sensing unit comprises at least one fourth sensor for measuring acoustic signals.

The feature of another possible embodiment of the invention is that it contains the upper body placed to connect the wrist configuration with the hand prosthesis.

The feature of another possible embodiment of the invention is that the wrist configuration includes at least one pulley provided to transmit the tendons it contains to the drive element.

The feature of another possible embodiment of the invention is that the wrist configuration includes a lower body configured to include the said pulley and drive element.

The feature of another possible embodiment of the invention is that the wrist configuration includes a joint placed between the lower body and the upper body to connect the lower body and the upper body to each other. Thus, it is ensured that the wrist can be moved within the specified degree of freedom.

The feature of another possible embodiment of the invention is that the wrist configuration is made of biocompatible material with three-dimensional printers. Thus, wrist movements can be easily performed.

### BRIEF DESCRIPTION OF THE DRAWINGS

FIG. 1 shows a representative view of a wrist configuration in an artificial hand system and multiple sensors provided to a sensing unit on an individual.

FIG. 2 shows a representative view of an artificial hand system.

FIG. 3 shows a representative view of the working scenario of an artificial hand system.

### Reference Numbers Given in the FIGS.

10 Artificial hand system
100 Sensing unit
   101 First sensor
   102 Second sensor
   103 Third sensor
   104 Fourth sensor
200 Processor unit
300 Database
400 Wrist configuration
   401 Lower body
      4011 Rotatory mechanism
         4012 Drive element
   402 Joint
   403 Upper body

### DETAILED DESCRIPTION OF THE EMBODIMENTS

In this detailed description, subject matter of the invention is explained with examples that will not have any limiting effect, for better understanding the subject matter.

The invention relates to an artificial hand system that can be controlled with an intelligent wrist configuration in order to reduce the physical and mental load/discomfort on the muscles of an individual using a hand prosthesis while using the said hand.

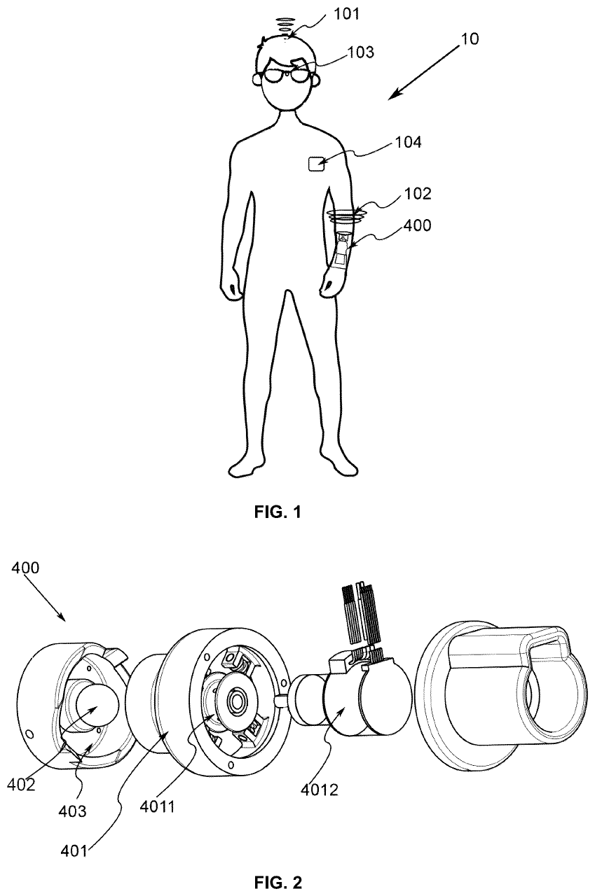

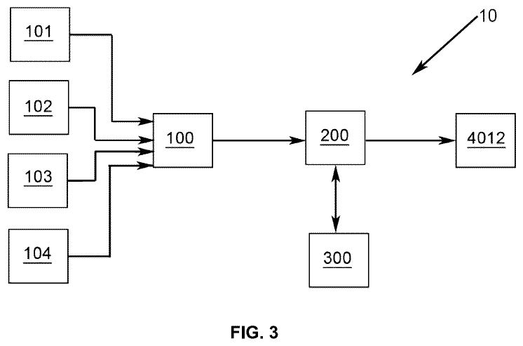

As shown in FIG. 1, said artificial hand system includes a wrist configuration to be placed between the individual's hand prosthesis and a specified amputation level. The said wrist configuration was provided to perform flexion/extension and ulnar/radial deviation movements in accordance with the human wrist. In a possible embodiment of the invention, it is preferred to use biocompatible materials with three-dimensional printers in the construction of the wrist configuration. Thus, wrist movements can be easily performed. The wrist configuration includes an upper body provided to be connected with the hand prosthesis. The wrist configuration also includes a lower body provided so that it remains between the upper body and the rest according to the amputation level. There is a joint placed between the said lower body and the upper body, which connects the lower body and the upper body. In a possible embodiment of the invention, it is preferred to use a spherical joint as the said joint. The wrist configuration is configured to allow the spherical joint to move with two degrees of freedom. The tendons that provide the movement of the wrist pass between the lower body and the upper body. There is at least one drive element provided to the lower body to ensure the movement of the tendons. In a possible embodiment of the invention, a motor etc. is used as the said drive element. There is a rotatory mechanism to connect the tendons extending from the upper body to the drive element in the lower body. Said rotatory mechanism can be a pulley, etc. The rotatory mechanism is used to feed the drive element by preventing the tendons from connecting directly to the drive element. In a possible embodiment of the invention, it is preferred to use a circular pulley. In an alternative embodiment of the invention, a star-shaped pulley is used.

The artificial hand system includes at least one sensing unit that enables the weight of said load to be sensed in order to lift a load. In a possible embodiment of the invention, the sensing unit comprises at least one first sensor for measuring electrical biological activity signals in the brain. When a person looks at a load, depending on the visual weight perception, signals related to the weight of the said load are formed in the brain according to previously learned information. The first sensor provides the measurement of said weight signals formed in the brain. The sensing unit also includes at least one second sensor for measuring the electrical biological activity signals resulting from muscle movement. Before a human muscle lifts a load, it contracts according to the load to be lifted. This is like a preparation method of the body. The second sensor provides the measurement of this contraction. The second sensor provides the detection of the weight of the load depending on the contractions. The sensing unit also includes a third sensor, which includes a visual sensor placed on the individual with the wrist configuration on his/her hand. In a possible embodiment of the invention, a camera etc. is used as the said third sensor. For example, a camera placed on the glasses allows the person to view the load they want to lift. The images are interpreted by using image processing and artificial intelligence learning methods. Thus, the weight of the load can be detected. The sensing unit further includes a fourth sensor including a sound sensor for measuring acoustic signals. After, the user sees a load the sound sensor is provided to say an audible expression such as heavy, light, medium heavy, etc. to the load that the user wants to lift. The fourth sensor provides the detection of the weight of the load by defining the audible expression.

There is a processor unit configured to receive the measured data via the sensing unit. There is at least one database provided to store the architectural details, coefficients and parameters of at least one model, which is obtained as a result of mathematically modeling the relationship between the previously received data and the weight of the load to be moved by the individual. The said database is provided to be associated with the processor unit. The processor unit provides the prediction of the weight depending on the information previously learned using the artificial learning method and recorded in the database of the data measured by the sensing unit. The processor unit provides the actuation of the drive element that provides the movement of the wrist configuration, depending on the detected weight situation. Said drive element is operated depending on the weight of the load. The operating phase of the drive element provides the adjustment of the stiffness of the wrist. The wrist is initially held in a reference position. In order to bring the wrist, whose drive element moves according to the weight of the load to be carried, to the reference position, the stiffness of the wrist is adjusted by stretching the tendons.

An exemplary working scenario of the invention is explained as follows;

The wrist configuration is placed on the arm of an individual who needs a bionic hand. When the individual using the wrist configuration moves her arm to carry a load, at least one data about the weight of the load is transmitted from the sensing unit to the processor unit. At least one of the data transmitted to the processor unit is provided by the first sensor. Said first sensor provides the measurement of the weight signals created by the brain according to the previously learned information when it sees the load. For example, when a person sees a pencil and thinks that the pencil is a lightweight object; the human brain provides the generation of a first data containing the information that the pencil is lightweight. The human brain can determine this information based on previous experiences. According to another example, when a large stone is seen, it is thought to be a heavy object. In this case, the human brain provides the generation of a second data containing the information that the stone is heavy. The first sensor provides the measurement of these weight signals in the brain.

At least another of the data transmitted to the processor unit is provided by the second sensor. The said second sensor enables the measurement of the amount of contraction created in the muscles according to the previously learned information as soon as the brain sees the load. For example, when we think that an object is lightweight, our muscles become less stiff. In this case, the first data containing the information that the object is lightweight is transmitted to the processor unit. When we think that the object is heavy, our muscles contract at the maximum level. In this case, the second data containing the information that the object is heavy is transmitted to the processor unit.

At least another of the data transmitted to the processor unit is provided by the third sensor. The third sensor provides the visualization of the object with an image sensor placed in such a way as to display the load to be carried by the person. The processor unit provides the interpretation of the data received from the image sensor by using image processing and artificial learning methods. The processor unit provides the prediction of the weights of the objects in the interpreted images by means of previously created models. For example, if the displayed object is a paper, the processor unit produces the first data indicating that the object is lightweight. If the displayed object is a full suitcase, the processor unit produces the second data indicating that the object is heavy.

At least another of the data transmitted to the processor unit is created by the fourth sensor. The fourth sensor provides the perception of the user's voice with a sound sensor placed on the person. When the user sees the object and says it is lightweight, the first data containing that the object is lightweight is produced. When the user sees the object and says it is heavy, the second data is produced.

The processor unit ensures that a weight data related to the weight of the object is received from at least one of the first sensor, second sensor, third sensor and fourth sensor included in the sensing unit. As a result of inputting the weight data taken from the processor unit sensing unit to the models stored in the database, the weight of the object is predicted. Depending on the weight of the object, the drive element is operated continuously and gradually. If the object is lightweight, less stiffness of the wrist is provided by ensuring that the drive element contracts the tendons less. If the object is heavy, more stiffness of the wrist is provided by ensuring that the drive element contracts the tendons more. In the event that the user realizes that the object, which she/he thinks as lightweight, is heavy when she/he takes the object in her/his hand, the drive element is provided to increase the wrist stiffness from the light stage to the heavy stage. By activating the drive element, the wrist is brought to the reference point.

Thus, the wrist is brought to the reference position before carrying the object by adjusting the stiffness of the wrist according to the weight of the object. Thus, it is ensured that the load is carried and managed with the wrist configuration, and the physical and mental effort caused by the extra work of healthy muscles is reduced.

In an exemplary embodiment of the invention, an intelligent wrist configuration is provided to be placed between a person's elbow and wrist. Said person is provided to carry a first object and a second object placed on a table. It is known that the said first object is an empty water bottle. It is known that the said second object is a full water bottle. If the person wants to move the first object on the table, a signal containing the information that the object is lightweight is provided according to the EEG signals received from the brain and the EMG signal received from the muscles. The processor unit provides classification of the signals received from the sensing unit with the model parameters stored in the database. The processor unit decides that the first object is lightweight as a result of classification. In this case, the processor unit ensures that the motor is actuated in the first stage. By actuating the motor in the first stage, it is ensured that the wrist, which is stiffened by one degree, is brought to the reference point.

In case the person wants to carry the second object, images are taken by the camera placed on the person's glasses. The images taken from the camera are transmitted to the processor unit. The processor unit provides the processing of the image taken by the camera. It detects that the interpreted image is a full water bottle. The processor unit detects that a full water bottle is in the heavy class. In this case, the processor unit provides the stretch of the tendons by actuating the motor in the second stage. By actuating the motor in the second stage, it is ensured that the wrist, which is stiffened by two degrees, is brought to the reference point.

Thus, while a person with an intelligent wrist configuration carries an object, it is ensured that most of the object is lifted by means of the intelligent wrist configuration. This ensures that the weight on the shoulder/arm muscles of the individual is reduced. Thus, it is ensured that the individual's sense of fatigue and the mental load that occurs when using prostheses are reduced.

The scope of protection of the invention is specified in the attached claims and it cannot be limited to what is explained in this detailed description for the sake of example. It is clear that a person skilled in the art can provide similar embodiments in the light of the above, without departing from the main theme of the invention.

What is claimed is:

1. An artificial hand system to reduce physical and mental discomfort in muscles of an individual using a hand prosthesis while using the hand prosthesis, comprising:
a wrist configuration, wherein a stiffness of the wrist configuration is controlled according to a weight of a load;
a sensing unit for sensing the weight of the load;
a processor unit for receiving data from the sensing unit; and
a drive element actuated by the processor unit;
wherein the processor unit is configured to:
receive at least one data related to the weight of the load from the sensing unit,
process at least one data about the weight taken from the sensing unit by methods comprising signal processing and machine learning,
detect a continuous and/or gradual relationship of processed data with the weight of the load,
generate an output signal containing weight information of the load as a result of a detected weight relationship, and
actuate the drive element to set the stiffness of the wrist configuration based on the output signal.

2. The artificial hand system according to claim 1, wherein the sensing unit comprises at least one first sensor for measuring biological activity signals in a brain.

3. The artificial hand system according to claim 1, wherein the sensing unit comprises at least one second sensor for measuring biological activity signals, wherein the biological activity signals occur due to muscle movement.

4. The artificial hand system according to claim 1, wherein the sensing unit comprises at least one third sensor for measuring visual signals.

5. The artificial hand system according to claim 1, wherein the sensing unit comprises at least one fourth sensor for measuring acoustic signals.

6. The artificial hand system according to claim 1, further comprising an upper body placed to connect the wrist configuration to the hand prosthesis.

7. The artificial hand system according to claim 1, wherein the wrist configuration comprises at least one pulley provided to transmit tendons of the wrist configuration to the drive element.

8. The artificial hand system according to claim 1, wherein the wrist configuration comprises a lower body, wherein the lower body comprises the pulley and the drive element.

9. The artificial hand system according to claim 1, wherein the wrist configuration comprises a joint placed between a lower body and an upper body to connect the lower body and the upper body to each other.

10. The artificial hand system according to claim 1, wherein the wrist configuration is made of biocompatible material with three-dimensional printers.
